(19) United States
(12) Patent Application Publication
(10) Pub. No.: US 2010/0234430 A1
(43) Pub. Date: Sep. 16, 2010

(54) METHODS AND PHARMACEUTICAL FORMULATIONS FOR PROTECTING PHARMACEUTICAL COMPOUNDS FROM ACIDIC ENVIRONMENTS

(76) Inventors: Rajneesh Taneja, Liberttyville, IL (US); Pramod Gupta, Gurnee, IL (US)

Correspondence Address: Steven F. Weinstock;Abbott Laboratories, D-377/AP6D, 100 Abbott Park Road, Abbott Park, IL, 60064-3500, US

(21) Appl. No.: 12/558,725

(22) Filed: Sep. 14, 2009

Related U.S. Application Data
(63) Continuation of application No. 09/955,801, filed on Sep. 19, 2001, now abandoned.

(51) Int. Cl. A61K 31/4439; A61P 1/04

(52) U.S. Cl. 514/338

(57) ABSTRACT

Methods and pharmaceutical compositions for protecting pharmaceutical compounds (or drugs) in acidic environments are provided. Methods of treatment using formulations capable of protecting pharmaceutical compounds in acidic environments are also provided. Formulations provided generally comprise a therapeutically effective amount of at least one pharmaceutical compound, and a pharmaceutically acceptable protectant. The pharmaceutically acceptable protectant of the invention generally comprises a water-soluble acid neutralizer, and a water-insoluble acid neutralizer.

### FIELD OF THE INVENTION

The present invention relates to pharmaceutical formulations and more particularly relates to pharmaceutical formulations that protect pharmaceutical compounds in acidic environments.

### BACKGROUND OF THE INVENTION

Many pharmaceutical compounds are susceptible to degradation in acidic environments. For example, certain antibiotics such as erythromycin; proton pump inhibitors (or “PPIs”) such as lansoprazole, or omeprazole; and pencreatin; are compounds that degrade in acidic environments and are therefore referred to a “acid labile”. Oral delivery of acid labile pharmaceutical compounds is challenging because the gastric pH is very acidic (typically between about pH 1.5 and 1.9). Additionally, the gastric volume of the stomach is typically between about 50 ml and 100 ml and is replenished at a gastric acid secretion rate of approximately 0 mM to 11 mm per hour. Moreover, the gastric retention time (the time a substance stays in the gastric environment) in a fasting state is generally about 30 to 60 minutes. Consumption of relatively small amounts of food cause increases in the gastric acid secretion rate and gastric acid retention time. Hence, under such conditions acid labile pharmaceuticals typically degrade and are not readily available for uptake without being protected.

For example, lansoprazole is a substituted benzimidaole that is an acid labile pharmaceutical compound that inhibits gastric acid secretions. The affect of gastric pH on the degradation of an acid-labile drug such as lansoprazole is conveyed in Table 1. The data shown in Table 1 was collected at 37 degrees C., wherein “K” reflects the first order degradation constant. The data presented in Table 1 demonstrates that lansoprazole is unstable in mildly acidic conditions wherein such acid-labile drugs undergo rapid acid-catalyzed degradation. Conversely, Table 1 also shows that lansoprazole remains relatively stable at neutral or alkaline pH's.

TABLE 1

| | Half life | | % Drug Remaining |
|---|---|---|---|
| pH | (hours) | K (hr.⁻¹) | After 30 Minutes |
| 5 | 0.52 | 1.33 | 51.37 |
| 6 | 3.4 | 0.20 | 90.30 |
| 7 | 18 | 0.04 | 98.10 |
| 8 | 37 | 0.02 | 99.10 |
| 9 | 78 | 0.01 | 99.57 |

Due to the pH sensitivity of acid labile drugs, they typically are administered in a form that protects the drug from the acidic gastric environment. Ideally, these drugs should reach the duodenum or upper small intestinal region in an intact, absorbable form, where the drug can be rapidly absorbed.

Enteric coating is probably the most popular method of protecting acid-labile drugs from gastric degradation. In enteric coating methods, either the drug particles or the dosage form is coated with a polymer that does not dissolve upon introduction to the low pH of the gastric environment, but does dissolve at a pH greater than 6, such as that found in the upper small intestine. Unfortunately, Enteric coated compositions are difficult to formulate as liquids, thus creating difficulty in administration to pediatric patients and/or patients having difficulty swallowing. Additionally, the pH of the gastric environment, the gastric acid secretion rate, and the gastric retention time are dependent upon a host of physiological factors that varies between individuals. Accordingly, the dissolution time for an enteric coating varies from recipient to recipient and may vary in the same recipient depending upon, for example, whether they ate prior to ingesting the composition.

Acid-labile drugs also have been protected from the acidic gastric environment of the stomach by neutralizing the pH of the gastric fluids prior to, or concomitantly with, administration of an acid-labile drug. Liquid formulations with the above purpose in mind have incorporated a neutralizer in combination with enterically and non-enterically coated drugs. However, such conventional methods generally use large dosages of acid neutralizer such as sodium bicarbonate, resulting in the production of stomach gases, and thus belching. Regrettably, production of stomach gases can be detrimental to individuals suffering from gastro-esophageal reflux disease (GERD). Obviously, this situation is particularly detrimental to patients taking PPI's for purposes of alleviating GERD.

While neutralizing compounds such as sodium bicarbonate are effective to neutralize the initial acidic state of the gastric environment, gastric retention in a non-fed state is about 30-60 minutes. As mentioned above, this retention time increases due to factors such as food consumption. Accordingly, it is critical that the acid neutralizer not only neutralize the initial pH of the gastric environment, but also maintain elevated pHs throughout the gastric retention period.

Additionally, PPI's, for example, typically do not provide relief of gastric distress until 1.5 to 2 hours after administration. Hence, in cases where relief of gastric distress is desired, such as when PPI's are taken, it would be advantageous to provide an increased pH until the therapeutic effect of the PPI is achieved. While relief from gastric acid irritation is usually achieved at a pH of around 3.5-4.0, it is nevertheless important to maintain the pH of the gastric environment at a higher pH than the patients comfort level for as long as possible to permit a PPI, for example, to enter the desired region of the digestive tract and achieve a therapeutic effect.

In light of the above, there is a need for dosage forms and methods for promoting and also maintaining a gastric pH that not only provides symptomatic relief but also provides an environment that does not rapidly degrade acid-labile drugs. Additionally, a need exists for methods and pharmaceutical compositions which avoid the difficulties associated with enterically coated formulations yet provides sufficient stability for either solid or liquid formulations.

### SUMMARY OF THE INVENTION

The present invention provides methods and formulations for protecting acid-labile pharmaceutical compounds in acidic environments.

Formulations provided herein generally comprise a therapeutically effective amount of an acid labile pharmaceutical compound and a water soluble acid neutralizer as well as a water insoluble acid neutralizer. The formulation may also include a gastric acid secretion stimulant and other therapeutically effective amounts of acid-labile or acid stable pharmaceutical compounds. Preferably, the formulations or pharmaceutical compounds included in the formulations are not enterically coated. Any of the above formulations can be administered to a patient in need of therapy for physiological disorders for which the pharmaceutical compounds are indicated.

Methods for protecting an acid-labile pharmaceutical compound from acidic environments are also provided. Generally, such methods comprise combining an acid-labile drug with a water soluble acid neutralizer as well as a water insoluble acid neutralizer.

### BRIEF DESCRIPTION OF THE FIGURES

FIG. 1 shows a line graph of pH versus time of gastric acid neutralization using a water insoluble neutralizing agent.

FIG. 2 shows a titration graph illustrating pH versus volume of neutralization suspension/solution added to 50 ml of simulated gastric fluid (SGF).

FIG. 3 shows another titration graph illustrating pH versus volume of neutralization suspension/solution added to 50 ml of SGF.

FIG. 4 illustrates a graph of pH variance as SGF is changed every 15 minutes to mimic initial and subsequent gastric secretions.

### DETAILED DESCRIPTION OF THE INVENTION

As previously mentioned, the present invention provides methods and formulations for protecting pharmaceutical compounds in acidic environments. Advantageously, the methods and formulations provided herein increase the pH in the environment of the acid-labile pharmaceutical compound to levels that are both comforting to a patient, but also to levels that are sufficient to protect an acid-labile pharmaceutical composition from degradation. Additionally, the formulations and methods increase pH levels and maintain elevated pH levels sufficiently to permit acid-labile pharmaceutical compounds to, for example, pass through the stomach and into the upper intestinal tract without substantial degradation. As a result, acid-labile pharmaceutical compounds are able to achieve their desired effect. Moreover, the present inventions ability to provide increased pH levels for extended periods also provides short term relief from ulcer aggravation that typically occurs at low pH levels. The aforementioned formulations can be provided in a non-enterically coated dosage form which makes these formulations easier to manufacture.

Formulations of the invention generally include a water-soluble acid neutralizer, a water-insoluble acid neutralizer, and a therapeutically effective amount of at least one acid-labile pharmaceutical compound. It has been surprisingly and unexpectedly discovered that the combination of acid neutralizers is capable of increasing the pH to a greater extent and maintaining the pH at increased levels for a greater time period than either of the acid neutralizers alone. The term “water soluble acid neutralizer” means any pharmaceutically acceptable compound or substance capable of increasing the pH of a solution that has a solubility of at least 1 gm in 100 ml, preferably at least 1 gm in 75 ml, and more preferably at least 1 gm in 30 ml. Examples of water-soluble acid neutralizers include, but are not limited to meglumine, sodium bicarbonate, sodium carbonate, sodium citrate, calcium gluconate, disodium hydrogen phosphate, dipotasium hydrogen phosphate, tripotasium phosphate, sodium tartarate, sodium acetate, calcium glycerophosphate, and preferably tromethamine, or any combination of the foregoing. The term “water-insoluble acid neutralizer” means any pharmaceutically acceptable compound or substance capable of increasing the pH of a solution that has a solubility less than 1 gm in 1,000 ml, preferably less than 1 gm in 5,000 ml, and more preferably less than 1 gm in 10,000 ml. Examples of water-insoluble acid neutralizers include, but are not limited to magnesium hydroxide, aluminum hydroxide, dihydroxy aluminum sodium carbonate, calcium carbonate, aluminum phosphate, aluminum carbonate, dihydroxy aluminum amino acetate, magnesium oxide, magnesium trisilicate, magnesium carbonate, and combinations of the foregoing.

The amount and ratio of the water-soluble acid neutralizer and water-insoluble acid neutralizer in a formulation generally does not depend upon the amount of the acid-labile drug administered and may vary widely to achieve a rapid and sustained pH increase sufficient to protect an acid-labile pharmaceutical compound from degradation. Exact amounts of the neutralizers employed is a matter of choice for those skilled in the art which can be determined empirically using experiments such as those provided in the examples below. For example, different amounts and proportions of the neutralizers may be tested in various amounts simulated gastric fluid and conditions to arrive at a desired effect. Generally, the quantity of water-soluble neutralizer in the formulation is between 50 mg and 1000 mg, preferably between 100 mg and 600 mg, and more preferably between 300 mg and 500 mg. The quantity of water-insoluble neutralizer in the formulation is typically between 100 mg and 1000 mg, preferably between 250 mg and 750 mg, and more preferably between 250 mg and 600 mg.

The combination of water-soluble and water-insoluble acid neutralizers is variously referred to herein as a “pharmaceutical protectant”. Preferably, the pharmaceutical protectant can elevate the pH of 50 ml of simulated gastric fluid (as shown below) above 7 within 20 minutes, more preferably within 15 minutes and most preferably within 10 minutes or less. Additionally, the pharmaceutical protectant typically can maintain the pH of simulated gastric fluid in simulated gastric conditions, such as those found in Example 5 below, above 3 for 30 minutes, preferably above 3 for 60 minutes, and more preferably above 3 for 90 minutes.

The combination of acid neutralizers mentioned above are particularly suited for protecting acid-labile pharmaceutical compounds from acid environments such as those found in the gastro-intestinal tract and in particular, the stomach. The phrase “acid-labile” refers to the tendency or potential for a moiety to alter, decompose, degrade, or otherwise become pharmacologically ineffective, due to the presence of the moiety in an acidic environment. The term pharmaceutical compound as used herein means drugs, prodrugs, or compounds otherwise indicated for animal use, as well as pharmaceutically acceptable salts and enantiomers of the foregoing. Examples of acid labile pharmaceutical compounds include, but are not limited to, certain antibiotics such as erythromycin; proton pump inhibitors (or “PPIs”) such as lansoprazole, or omeprazole; and pencreatin. PPIs are particularly preferred acid labile pharmaceutical compounds for use in the present invention. PPIs are well known substituted benzimidazoles such as omeprazole, lansoprazole, pantoprazole, pariprazole, and leminoprazole.

A presently preferred proton pump inhibitor is lansoprazole, shown below.

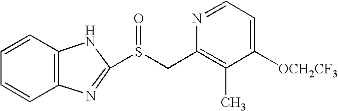

It will be understood that in addition to acid-labile pharmaceutical compounds, the formulations of the present invention may also include other pharmaceutical compounds that are not acid labile which may include, for example, non-steroidal anti-inflamatory drugs (“NSAIDs”), antibiotics, and the like. Combinations of acid labile pharmaceutical compounds may also be employed in accordance with the present invention.

In embodiments of the invention which include proton pump inhibitors, such as, for example, lansoprazole, the formulation may further comprise an ingredient to enhance the effectiveness of the PPI. In particular, while PPIs should be protected from an acidic environment in the gastrointestinal tract, they need an acidic environment in the targeted parietal cells. Proton pump inhibitors are substantially devoid of acid inhibiting properties at a neutral pH. Hence, once PPIs are delivered to the parietal cells via systemic blood circulation, conditions inside the parietal cells of the stomach need to be acidic to protonate the PPI such that it is converted the active metabolite capable of neutralizing acidic conditions. Therefore, compounds that would cause acidic conditions in parietal cells would be considered a “PPI enhancer”. Some common food products such as caffeine, beer, and milk, stimulate gastric secretions and cause conditions in the parietal cells to acidify. Gastrin release and acid secretion in parietal cells is also stimulated by oral ingestion of calcium salts such as, for example, calcium carbonate, calcium acetate, and calcium citrate. Peptides and amino acids also stimulate a similar parietal cell response. Hence, sodium caseinate, casein, whey protein, taurine, alanine, tryptophan, lysine, methionine, phenylalanine, threonine, valine, leucine, argenine, glycine, serine, histadine, cystine, tyrosine, proline, and histadine are also examples of PPI enhancers. Caffeine is a further example of a PPI enhancer. When employed, the quantity of the above PPI enhancers is typically less than that required for purposes of gastric neutralization, typically less than 250 mg, and preferably less than 225 mg.

As mentioned above, pharmaceutical compounds can be utilized in the form of pharmaceutically acceptable salts derived from inorganic or organic acids. The phrase “pharmaceutically acceptable” as used herein includes moieties or compounds that are, within the scope of sound medical judgment, suitable for use in contact with the tissues of humans and lower animals without undue toxicity, irritation, allergic response, and the like, and are commensurate with a reasonable benefit/risk ratio.

For example, pharmaceutically acceptable salts are well known in the art. Such salts can be prepared in situ during the final isolation and purification of the compounds of the invention, or separately by reacting a free base function with a suitable organic acid. Representative acid addition salts include, but are not limited to acetate, adipate, alginate, citrate, aspartate, benzoate, benzenesulfonate, bisulfate, butyrate, camphorate, camphor sulfonate, digluconate, glycerophosphate, hemisulfate, heptanoate, hexanoate, fumarate, hydrochloride, hydrobromide, hydroiodide, 2-hydroxyethansulfonate (isothionate), lactate, maleate, methane sulfonate, nicotinate, 2-naphthalene sulfonate, oxalate, palmitoate, pectinate, persulfate, 3-phenylpropionate, picrate, pivalate, propionate, succinate, tartrate, thiocyanate, phosphate, glutamate, bicarbonate, p-toluenesulfonate, and undecanoate.

Further, basic nitrogen-containing groups can be quaternized with such agents as lower alkyl halides such as methyl, ethyl, propyl, and butyl chlorides, bromides, and iodides; long chain halides such as decyl, lauryl, myristyl, and stearyl chlorides, bromides, and iodides; arylalkyl halides like benzyl and phenethyl bromides and others. Water or oil-soluble or dispersible products are thereby obtained.

Examples of acids which can be employed to form pharmaceutically acceptable acid addition salts include such inorganic acids as hydrochloric acid, hydrobromic acid, sulphuric acid, and phosphoric acid, and such organic acids as oxalic acid, maleic acid, succinic acid, and citric acid.

Basic addition salts can be prepared in situ during the final isolation and purification of compounds of this invention by reacting a carboxylic acid-containing moiety with a suitable base such as the hydroxide, carbonate, or bicarbonate of a pharmaceutically acceptable metal cation or with ammonia or an organic primary, secondary, or tertiary amine. Pharmaceutically acceptable salts include, but are not limited to, cations based on alkali metals or alkaline earth metals such as lithium, sodium, potassium, calcium, magnesium, and aluminum salts, and the like, and nontoxic quaternary ammonia and amine cations including ammonium, tetramethylammonium, tetraethylammonium, methylammonium, dimethylammonium, trimethylammonium, triethylammonium, diethylammonium, and ethylammonium, amongst others. Other representative organic amines useful for the formation of base addition salts include ethylenediamine, ethanolamine, diethanolamine, piperidine, piperazine, and the like.

Formulations of the invention can be used in combination with virtually any pharmaceutical compound, such as those mentioned above, for treatment of almost any physiological and/or psychological disorders for which the pharmaceutical compounds are indicated. In the preferred use of combining a pharmaceutically acceptable protectant of the invention with an acid-labile non-enteric coated PPI, such acid-resistant combinations can be used for the treatment of various gastro-intestinal conditions. Exemplary gastro-intestinal conditions include “gastric acid disorders”, which herein include, but are not limited to, active duodenal ulcers, gastric ulcers, gastro-esophageal reflux disease (GERD), severe erosive esophagitis, poorly responsive systematic GERD, and pathological hyper-secretory conditions such as Zollinger Ellison Syndrome, among others. Gastric acid disorders also include disorders caused by imbalances between acid and pepsin production, known in the art as “aggressive factors”, and mucus, bicarbonate, and prostaglandin production, known in the art as “defensive factors”.

Hence, the invention also includes methods for treating physiological and psychological disorders comprising the step of orally administering to a patient in need of such treatment a therapeutically effective amount of at least one pharmaceutical compound, and preferably a acid-labile pharmaceutical compound, formulated with a water soluble neutralizer and a water insoluble neutralizer and, optionally, a PPI enhancer and/or other pharmaceutical compound.

The phrase “therapeutically effective amount” as used herein means a sufficient amount of, for example, the composition, compound, or formulation necessary to treat the desired disorder, at a reasonable benefit/risk ratio applicable to any medical treatment. As with other pharmaceuticals, it will be understood that the total daily usage of a pharmaceutical composition of the invention will be decided by a patient's attending physician within the scope of sound medical judgment. The specific therapeutically effective dose level for any particular patient will depend upon a variety of factors including the disorder being treated and the severity of the disorder; activity of the specific compound employed; the specific composition employed; the age, body weight, general health, sex and diet of the patient; the time administration, route of administration, and rate of excretion of the specific compound employed; the duration of the treatment; drugs used in combination or coincidental with the specific compound employed; and other factors known to those of ordinary skill in the medical arts. For example, it is well within the skill of the art to start doses of the compound at levels lower than required to achieve the desired therapeutic effect and to gradually increase the dosage until the desired effect is achieved.

Formulations of the invention are administered and dosed in accordance with sound medical practice, taking into account the clinical condition of the individual patient, the site and method of administration, scheduling of administration, and other factors known to medical practitioners.

Therapeutically effective amounts for purposes herein thus can readily be determined by such considerations as are known in the art. The amount must be affective to achieve improvement, including but not limited to, raising of gastric pH, reduced gastrointestinal bleeding, reduction in the need for blood transfusions, improved survival rate, more rapid recovery, and/or improvement/elimination of symptoms and other indicators as are selected as appropriate measures by those skilled in the art.

Formulations provided herein may also contain other well known pharmaceutically acceptable ingredients such as carriers, diluents, excipients, fillers and the like. The formulations provided herein can be administered in either a solid or liquid dosage form. Solid dosage forms of the invention for oral administration generally are fabricated in a similar manner to the tablets of the examples below. Similarly, liquid dosage forms of the invention for oral administration can be pharmaceutically acceptable emulsions, solutions, suspensions, syrups, and elixirs. In addition to the active compounds, the liquid dosage forms may contain inert diluents commonly used in the art such as water or other solvents, solubilizing agents and emulsifiers such as ethyl alcohol, isopropyl alcohol, ethyl carbonate, ethyl acetate, benzyl alcohol, benzyl benzoate, propylene glycol, 1,3-butylene glycol, dimethyl formamide, oils (in particular, cottonseed, goundnut, corn, germ, olive, castor, and/or sesame oils), glycerol, tetrahydrofurfuryl alcohol, polyethylene glycols and fatty acid esters of sorbitan and mixtures thereof.

Besides inert diluents, oral compositions of the invention may also include adjuvants such as wetting agents, emulsifying and suspending agents, sweetening agents, flavoring agents, and/or perfuming agents.

Compositions of the invention can be manufactured by utilizing an acid-labile and/or acid-stable pharmaceutical compound in the form of granules and/or powder. Alternatively, micronized acid-labile pharmaceutical compositions can be used in place of the granules or powder. Micronization is utilized in order to produce a particle having a smaller diameter in relationship to the granules. Since the dissolution rate of acid-labile pharmaceutical compositions of the invention is generally directly proportional to, among other factors, the surface area of the composition particle, a reduction in particle size increases the amount of exposed surface area and, thus, increases the dissolution rate.

Although micronization results in increased exposed surface area causing particle aggregation, which can negate the benefit of micronization and is an expensive manufacturing step, micronization of the proton pump inhibitor does present a significant benefit of increasing the dissolution rate of relatively water-insoluble drugs, e.g. omeprazole.

Examples are provided below to describe preferred embodiments and/or utilities of the invention. Such examples are not meant to limit the invention.

### EXAMPLES

For the following examples, simulated gastric fluid (“SGF”) was made by dissolving 2.0 gm of sodium chloride and 3.2 gm of purified pepsin (derived from porcine stomach mucosa) having an activity of 800 to 2500 units per mg of protein, in 7.0 mL of hydrochloric and sufficient water to make a 1000 mL solution. The solution had a pH of 1.2.

The so-called “carbicarb” dry powder mixture employed in the following examples, was created by transferring 46.93 g of sodium carbonate to a suitable container and adding 37.17 g of sodium bicarbonate. The powders were mixed by shaking.

### Example 1

### Testing Fast Acting/Water Soluble Neutralizers

Acid neutralization tests were conducted to identify chemical compositions which demonstrate soluble (or fast-acting) acid neutralizers. 840 mg of each of magnesium hydroxide, tromethamine, and carbicarb were separately compressed into tablets utilizing a carver press having a 13 mm die at a 1 second dwell time and 500 pounds of compression force. Also, a first mixture containing 420 mg of magnesium hydroxide and 420 mg of tromethamine was also compressed into tablet-form using the method described above. Additionally, a second mixture containing 420 mg of magnesium hydroxide and 420 mg of carbicarb was compressed into tablet-form as previously described.

Each of the five different preparations was tested separately in the following manner. Tablets of each preparation were placed in a USP dissolution apparatus basket which was then attached to a spindle. The stirring component of the apparatus was set to rotate the basket at a speed of approximately 75 rpm. The loaded, rotating basket was then immersed into a beaker containing 50 ml of SGF and 50 ml of distilled de-ionized water. The pH of the medium in the beaker was monitored continuously throughout the test procedure utilizing a micro pH electrode. The time required for the pH in the beaker to rise to 7 with the different 10 tablets is presented in Table 2.

TABLE 2

| | Time to Raise the pH of SGF to 7 |
|---|---|
| Tablet Composition | at 37 degrees C. |
| Tromethamine (TRIS) 840 mg | 75 seconds |
| Magnesium hydroxide 840 mg | 565 seconds |
| Carbicarb 840 mg | 200 seconds |
| Tromethamine 420 mg + | 185 seconds |
| Magnesium hydroxide 420 mg | |
| Carbicarb 420 mg + | 270 seconds |
| Magnesium hydroxide 420 mg | |

### Example 2

### pH Increase Using Water Insoluble Acid Neutralizers

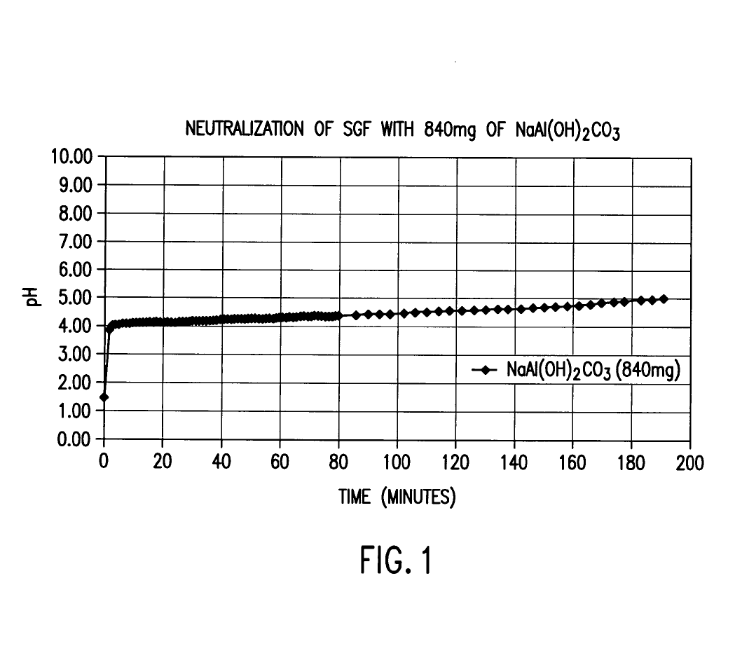

840 mg of calcium carbonate or dihydroxyaluminum sodium carbonate were added to a beaker containing 100 ml of modified simulated gastric fluid. The contents of the beaker were gently stirred and the pH of the contents was monitored constantly. pH observations were recorded for 20 minutes and are represented in Table 3. The pH of the dihydroxyaluminum sodium carbonate solution was measured for additional time and these measurements are presented graphically in FIG. 1.

TABLE 3

| | pH of SGF/DI-H₂O with | |
|---|---|---|
| Time | dihydroxyaluminum sodium | pH of SGF/DI-H₂O with 840 mg |
| (minutes) | carbonate added | calcium carbonate added |
| 0 | 1.51 | 1.51 |
| 1 | 3.82 | 3.1 |
| 2 | 4.04 | 5.31 |
| 3 | 4.08 | 5.56 |
| 4 | 4.09 | 5.67 |
| 5 | 4.10 | 5.74 |
| 6 | 4.11 | 5.81 |
| 7 | 4.12 | 5.89 |
| 8 | 4.12 | 5.98 |
| 9 | 4.13 | 6.06 |
| 10 | 4.13 | 6.14 |
| 11 | 4.13 | 6.23 |
| 12 | 4.14 | 6.31 |
| 13 | 4.14 | 6.38 |
| 14 | 4.14 | 6.45 |
| 15 | 4.15 | 6.52 |
| 16 | 4.15 | 6.59 |
| 17 | 4.15 | 6.65 |
| 18 | 4.15 | 6.72 |
| 19 | 4.16 | 6.77 |
| 20 | 4.16 | 6.83 |

As shown in FIG. 1 and Table 3, both calcium carbonate and dihydroxyaluminum sodium carbonate, by themselves, failed to raise the pH of modified simulated gastric fluid higher than 7 during the time the pH was measured.

### Example 3

### Titration of Acidic Samples with Separate Water Soluble and Water Insoluble Neutralizers

Several combinations of acid neutralizers were compared to determine neutralization properties of each of the combinations as a function of pH versus volume of the acid neutralizer(s) utilized.

Tromethamine (variously referred to as “TRIS”) and magnesium hydroxide, Mg(OH)₂, were mixed with each other in a variety of proportions shown in Table 4. Each of the different tromethamine/magnesium hydroxide mixtures was added to distilled water until a 10% suspension resulted. Two 10% solutions of carbicarb were also prepared as shown by Samples 5 and 6 of Table 4.

TABLE 4

| | Carbicarb | Tromethamine | Mg(OH)₂ | CaCO₃ |
|---|---|---|---|---|
| Sample # | (mg) | (mg) | (mg) | (mg) |
| 1 | — | 300 | 400 | 100 |
| 2 | — | 350 | 350 | 100 |
| 3 | — | 200 | 500 | 100 |
| 4 | — | 100 | 600 | 100 |
| 5 | 700 | — | — | 100 |
| 6 | *700 | — | — | *100 |

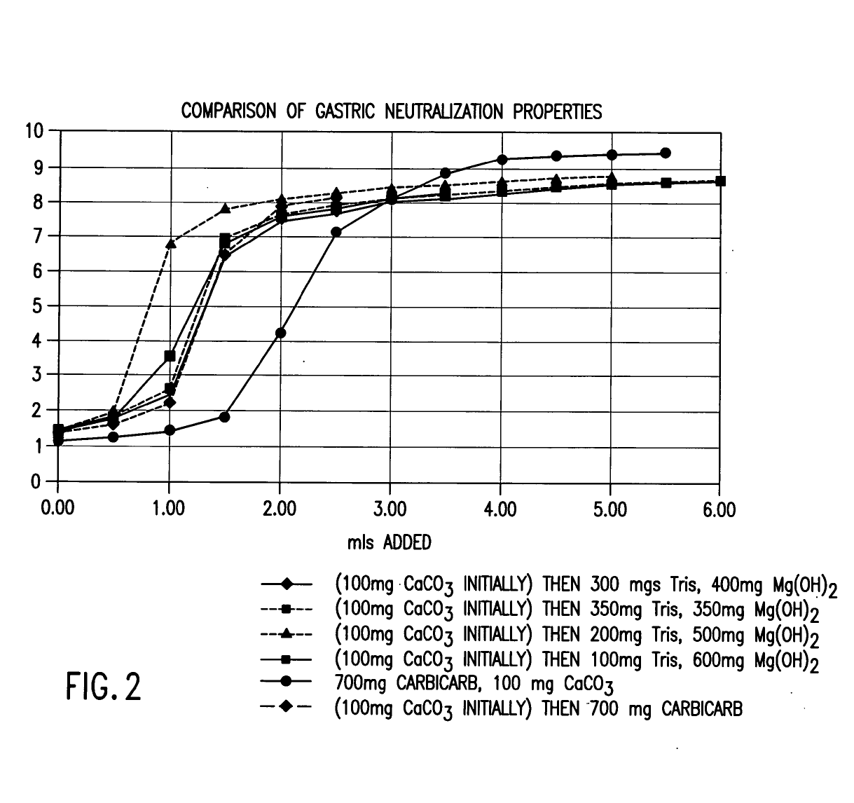

With one exception (Sample 6), in each portion of the neutralization tests 100 mg of calcium carbonate, CaCO₃, was initially added to 50 ml of SGF in a glass beaker while stirring. The beaker contents were then titrated against the different 10% antacid suspensions. In sample 6, the 100 mg of calcium carbonate was mixed with 700 mg of carbicarb before adding the mixture to the 50 ml of SGF. The minimum total quantity of antacid mixture, and the quantities of individual antacids comprising each respective mixture, required for raising the pH of the SGF to higher than 7 are graphically illustrated in FIG. 2.

As illustrated by FIG. 2, all combinations raised the pH of the SGF above 7 after addition of approximately 2.5 ml of the 10% solutions. The minimum total quantity of neutralizing mixture, including the quantities of the individual neutralizers, necessary to raise the pH of SGF to higher than 7 is reflected in Table 5 below.

TABLE 5

| | | Trometh- | | | | |
|---|---|---|---|---|---|---|
| | Carbicarb | amine | Mg(OH)₂ | CaCO₃ | Total | |
| Sample # | (mg) | (mg) | (mg) | (mg) | (mg) | pH |
| 1 | — | 60 | 80 | 100 | 240 | 7.53 |
| 2 | — | 70 | 70 | 100 | 240 | 7.62 |
| 3 | — | 30 | 75 | 100 | 205 | 7.80 |
| 4 | — | 20 | 120 | 100 | 240 | 7.61 |
| 5 | 140 | — | — | 100 | 240 | 7.93 |

### Example 4

### Titration of Acidic Samples with Combined Water Soluble and Water Insoluble Neutralizers

Example 3 was effectively duplicated except in this example the calcium carbonate CaCO₃, was added to the respective 10% suspensions of the respective samples before titrating the respective sample suspensions into the 50 ml of SGF. The amounts of each of the sample preparations is reflected in Table 6 below.

TABLE 6

| | Tromethamine | Mg(OH)₂ | CaCO₃ |
|---|---|---|---|
| Sample # | (mg) | (mg) | (mg) |
| 1 | 300 | 400 | 100 |
| 2 | 350 | 350 | 100 |
| 3 | 200 | 500 | 100 |
| 4 | 100 | 600 | 100 |

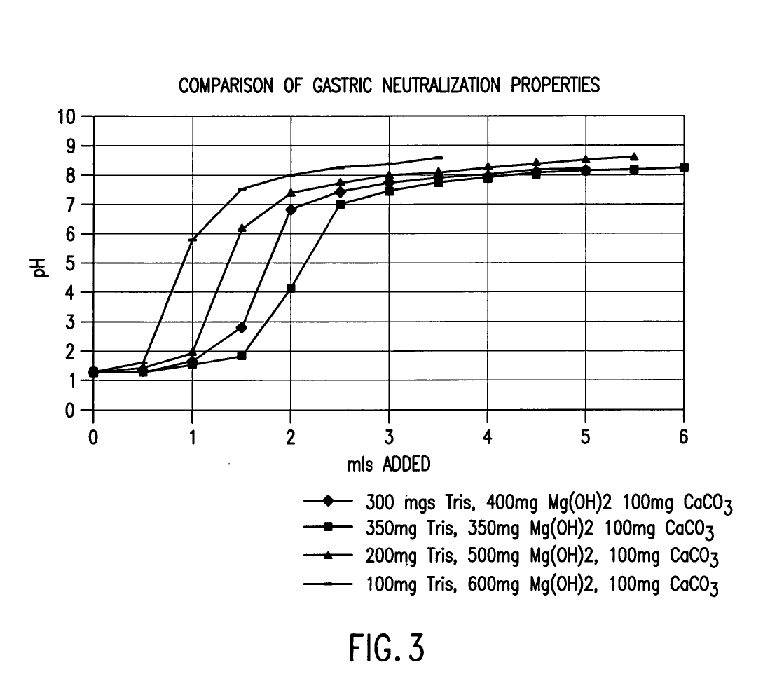

The minimum total quantity of antacid mixture, and the quantities of individual antacids comprising each respective mixture, required for raising the pH of the SGF to higher than 7 are graphically illustrated in FIG. 3.

As illustrated by FIG. 3, similarly to Example 3, all combinations raised the pH of the SGF above 7 after addition of approximately 2.5 ml of the 10% solutions. The minimum total quantity of neutralizing mixture, including the quantities of the individual neutralizers, necessary to raise the pH of SGF to higher than 7 is reflected in Table 7 below. Also included in Table 7 is the data from the procedural exception sample, Sample 6, of Table 4 above.

TABLE 7

| | | Trometh- | | | | |
|---|---|---|---|---|---|---|
| | Carbicarb | amine | Mg(OH)₂ | CaCO₃ | Total | |
| Sample # | (mg) | (mg) | (mg) | (mg) | (mg) | pH |
| 1 | — | 75 | 100 | 25 | 200 | 7.51 |
| 2 | — | 105 | 105 | 30 | 240 | 7.45 |
| 3 | — | 40 | 100 | 20 | 160 | 7.43 |
| 4 | — | 15 | 90 | 15 | 120 | 7.55 |
| 5 | 175 | — | — | 25 | 200 | 7.12 |

While at least approximately 240 mg of any of Samples 1-4 of Table 7 and Sample 6 of Table 4 effect a pH change on SGF to that above 7, Sample 4 effected the highest resultant pH, as well as the lowest total amount need to effect such a change, of the five samples illustrated on Table 7.

### Example 5

### Activity of Pharmaceutical Protectant in Simulated Gastric Conditions

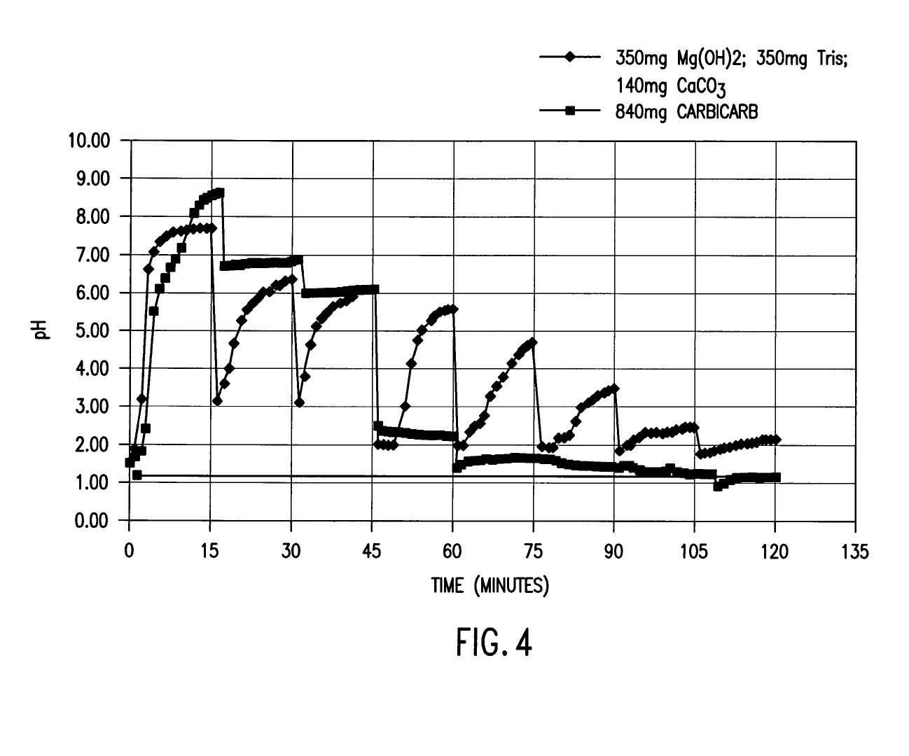

Acid neutralization tests were conducted to contrast buffer functioning of acid neutralizing components of the invention to that of carbicarb. A first sample contained 350 mg of magnesium hydroxide, 350 mg of tromethamine, and 140 mg of calcium carbonate. A second sample was utilized as a control and contained 840 mg of carbicarb. Both samples were pressed into tablets using a Carver Pellet Press (500 lb pressure, 1 second dwell time) and individually placed in a dissolution basket (Van Kel). Both baskets were lowered into separate beakers, each beaker containing 100 ml of SGF. The baskets were rotated at approximately 75 rpm and the solutions containing each sample were constantly monitored. 5 ml of the beaker contents were removed every 15 minutes utilizing a syringe with a 70 μm full flow filter attached to the syringe tip. 35 ml of fresh modified simulated gastric fluid was added back to the beaker. This process was repeated for 120 minutes. The results are presented FIG. 4.

As shown in FIG. 4, there is a gradual decline in the pH with each fluid replacement. In the case of carbicarb the pH decline after the 4^{th} fluid replacement is dramatic. Carbicarb and tromethamine are water-soluble acid neutralizers and they are partially removed from the beaker after each fluid replacement. Magnesium hydroxide and calcium carbonate are water insoluble and therefore they are not removed with each fluid replacement. This is similar to what is expected to occur in-vivo. The combination insoluble and soluble acid neutralizer shows better pH recovery and higher buffering capacity.

### Example 6

### Activity of Pharmaceutical Protectants

250 mg of calcium carbonate was mixed and blended with different proportions of tromethamine and magnesium hydroxide so that the total weight of the powder mixtures was either 800 mg or 700 mg. The constituents of the different powder blends are described in Table 8. 100 ml of SGF was placed in 7 water-jacketed beakers that were connected to a water bath set at 37.0° C. The powder blends were separately transferred to the beakers containing SGF with gentle stirring (Magnetic Stir Plate—Fisher Scientific) and allowed to mix for 10 minutes. PH of the beaker contents were recorded after the initial addition of the powder blend. After 10 minutes, an additional 5 ml of SGF was added to the beakers and mixed for 2 minutes at which point the pH was recorded. This process of adding 5 ml SGF was repeated until the pH of the beaker contents dropped below 6.0. Initial pH after the powder blend addition, pH 10 minutes after the powder blend addition, time required to raise the pH of SGF above 7.0, and the total volume of SGF required to lower the pH to below 6 are shown in Table 8.

All of the powder blends required 120 to 175 ml of Simulated Gastric Fluid (SGF) to attain a pH below 6.0. The intragastric fasting volume for adults (un-stimulated) is 24 ml (Reference: Geigy Scientific Tables). The basal flow rate of gastric juice in adults is 79.7 ml per hour (Reference: Geigy Scientific Tables). The published volume of gastric fluid is significantly lower than the volume of SGF used in this in-vitro example. Hence, it can be concluded that a combination of an acid-labile drug and a pharmaceutical protectant of the invention should be capable of offering faster onset of gastric acid neutralization and longer duration of pH control as compared to an acid-labile pharmaceutical compound alone.

TABLE 8

| | | | | | | Initial pH | pH 10 | Time to | |
|---|---|---|---|---|---|---|---|---|---|
| | | | | | Total | After | minutes | raise SGF | VolSGF |
| ID | Carbicarb | Tromethamine | Mg(OH)₂ | CaCO₃ | wt. | antacid | post antacid | pH to >7 | to pH 6.0 |
| No. | (mg) | (mg) | (mg) | (mg) | (mg) | addition | addition | (sec) | (ml) |
| A | 0 | 300 | 250 | 250 | 800 | 5.32 | 7.26 | 210 | 175 ml |
| B | 0 | 250 | 300 | 250 | 800 | 5.35 | 7.22 | 210 | 175 ml |
| C | 0 | 200 | 350 | 250 | 800 | 4.21 | 7.46 | 210 | 166 ml |
| D | 0 | 150 | 400 | 250 | 800 | 5.67 | 7.52 | 300 | 154 ml |
| E | 0 | 100 | 450 | 250 | 800 | 5.77 | 7.27 | 420 | 125 ml |
| F | 0 | 100 | 350 | 250 | 700 | 5.47 | 7.44 | 660 | 120 ml |
| G | 0 | 150 | 300 | 250 | 700 | 5.29 | 7.28 | 420 | 120 ml |

### Example 7

### Stability of Pharmaceutical Protectants

Lansoprazole granulations containing magnesium hydroxide and calcium carbonate, and tromethamine were prepared, tested for potency and evaluated for stability in simulated gastric fluid. The same granulations were tested again after 27 days room temperature storage (protected from light). In addition, suspension formulations were prepared and evaluated for stability at initial, day 14, and day 27 intervals for samples stored at both room temperature and refrigerated conditions.

Magnesium Hydroxide (Mallinckrodt), Sodium Bicarbonate (ACS grade, Fisher), Tromethamine, USP (Sigma), and Calcium Carbonate (ACS grade, Fisher) were mixed together with lansoprazole (Takeda Chemicals) in the proportions as described in Table 9. Sucrose, NF (Fisher) was dissolved in purified water (Fisher) to form a 60% solution. Sucrose solution was added to the powder mixture and triturated so that a wet coherent mass resulted. This coherent mass was passed through a size 10 sieve and the sieved granules were dried at 45° C. overnight. The dried granules were passed through the size 10 sieve again.

In a 100 ml glass beaker granules equivalent to 30 mg of lansoprazole were added to 50 ml of simulated gastric fluid USP and gently stirred. After 5 minutes 5 ml of 2 N sodium hydroxide was added to the beaker in order to freeze any further degradation of lansoprazole. The same procedure was repeated but 2 N sodium hydroxide (Certified grade, Fisher) was added after 60 minutes instead of 5 minutes. The contents of the 2 beakers were analyzed by a stability indicating HPLC procedure. There was less than 1% degradation of lansoprazole observed with the current formulation when it was added to 50 ml of simulated gastric fluid USP.

Granules containing about 300 mg lansoprazole were kept in a closed container at room temperature (22° C.±2° C.). After for 27 days, these samples were examined for any signs of physical changes and they were also analyzed by a stability indicating HPLC procedure. Lansoprazole was found to be stable in the granules prepared by the formulation described in Table 9.

TABLE 9

| | Formula |
|---|---|
| Ingredients | mg/dose |
| Lansoprazole | 30 |
| Magnesium Hydroxide | 350 |
| Calcium Carbonate | 140 |
| Sucrose* | 120 |
| Tromethamine | 350 |

### Example 8

### Stability of Pharmaceutical Protectant

The same procedure for granulation as described in Example 7 was repeated with the exception that a size 20 sieve was used instead of size 10 sieve. The resulting granules were used for making suspension formulation. An amount of granules for suspension, equivalent to 300 mg of lansoprazole (10 doses) was transferred into a 100-mL volumetric flask. 10 gm of flavor blend was added to the volumetric flask. Sufficient quantity of Purified water (Fisher) was then added to make up the volume up to 100 ml.

Part of the suspension was kept in the refrigerator (4° C.) and another part was kept at room temperature (22° C.±2° C.) in closed containers. After for 14 and 27 days, these samples were examined for any signs of physical changes and they were also analyzed by a stability indicating HPLC procedure. Lansoprazole was found to be stable in the suspension formulation under refrigeration and at room temperature storage

Those skilled in the art will now see that certain modifications can be made to the compositions and methods herein disclosed with respect to the herein described embodiments, without departing from the spirit of the instant invention. And while the invention has been described above with respect to the preferred embodiments, it will be understood that the invention is adapted to numerous rearrangements, modifications, and alterations, and all such arrangements, modifications, and alterations are intended to be within the scope of the appended claims.

What is claimed is:

1. A pharmaceutical formulation comprising:
(a) a therapeutically effective amount of at least one pharmaceutical compound; and
(b) a pharmaceutically acceptable protectant comprising
(i) a water-soluble acid neutralizer; and
(ii) a water-insoluble acid neutralizer.

2. The formulation of claim 1 wherein the pharmaceutical compound is acid-labile.

3. The formulation of claim 2 wherein the pharmaceutical compound is a proton pump inhibitor.

4. The formulation of claim 3 wherein the pharmaceutical compound is lansoprazole, an enantiomer of lansoprazole, or a pharmaceutical salt thereof.

5. The formulation of claim 1 wherein the water-soluble acid neutralizer is selected from tromethamine, meglumine, sodium bicarbonate, sodium carbonate, and combinations of tromethamine, meglumine, sodium bicarbonate, and sodium carbonate.

6. The formulation of claim 1 wherein the water-insoluble acid neutralizer is selected from the group consisting of magnesium hydroxide, aluminum hydroxide, dihydroxy aluminum sodium carbonate, calcium carbonate, and combinations of magnesium hydroxide, aluminum hydroxide, dihydroxy aluminum sodium carbonate, and calcium carbonate.

7. The formulation of claim 3 further comprising a proton pump inhibitor enhancer.

8. The formulation of claim 7 wherein the pharmaceutical compound is lansoprazole, an enantiomer of lansoprazole, or a pharmaceutical salt thereof.

9. A pharmaceutical formulation for treating gastric acid disorders, said pharmaceutical composition comprising:
(a) a therapeutically effective amount of a proton pump inhibitor; and
(b) a pharmaceutically acceptable protectant surrounding said proton pump inhibiting composition, said pharmaceutically acceptable protectant including
(i) a water-soluble acid neutralizer, and
(ii) a water-insoluble acid neutralizer.

10. A pharmaceutical composition as in claim 9, the water-soluble acid neutralizer comprising one or more of tromethamine, meglumine, sodium bicarbonate, and sodium carbonate.

11. A formulation of claim 9 wherein the water-soluble acid neutralizer is selected from tromethamine, meglumine, sodium bicarbonate, sodium carbonate, and combinations of tromethamine, meglumine, sodium bicarbonate, and sodium carbonate.

12. The formulation of claim 9 wherein the water-insoluble acid neutralizer is selected from the group consisting of magnesium hydroxide, aluminum hydroxide, dihydroxy aluminum sodium carbonate, calcium carbonate, and combinations of magnesium hydroxide, aluminum hydroxide, dihydroxy aluminum sodium carbonate, and calcium carbonate.

13. The formulation of claim 9 wherein the proton pump inhibitor is lansoprazole, an enantiomer of lansoprazole or a pharmaceutically acceptable salt thereof.

14. A method for protecting a pharmaceutical compound from gastric fluid degradation comprising the steps of: combining a therapeutically effective amount of at least one pharmaceutical compound, with a pharmaceutically acceptable protectant to thereby protect the pharmaceutical compound, wherein the pharmaceutically acceptable protectant comprises a water-soluble acid neutralizer and a water-insoluble acid neutralizer.

15. The method of claim 14 wherein the pharmaceutical compound is acid labile.

16. The method of claim 15 wherein pharmaceutical compound is lansoprazole, an enantiomer of lansoprazole, or a pharmaceutical salt thereof, including selecting at least one of magnesium hydroxide, aluminum hydroxide, and calcium carbonate as the water-insoluble acid neutralizer.

17. A method for treating a physiological disorder comprising administering a pharmaceutically acceptable amount of the formulation of claim 1.

18. The method of claim 17 wherein the pharmaceutical compound is acid-labile.

19. The method of claim 18 wherein the pharmaceutical compound is a proton pump inhibitor.

20. The method of claim 19 wherein the pharmaceutical compound is lansoprazole, an enantiomer of lansoprazole, or a pharmaceutical salt thereof.

21. The method of claim 20 wherein the formulation further comprising a proton pump inhibitor enhancer.
